(19) United States
(12) Patent Application Publication
(10) Pub. No.: US 2006/0004527 A1
(43) Pub. Date: Jan. 5, 2006

(54) Methods, systems and computer readable media for identifying dye-normalization probes

(76) Inventors: Nicholas M. Sampas, San Jose, CA (US); Jayati Ghosh, San Jose, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/884,731

(22) Filed: Jul. 1, 2004

(51) Int. Cl. C12Q 1/68; G06F 19/00; G01N 33/48

(52) U.S. Cl. 702/20

(57) ABSTRACT

Methods, systems and computer readable media for identifying dye-normalization probes. Intensity signals read from probes on a set of existing multi-channel microarrays are provided. The intensity signals are combined from each channel for each probe to generate a combined signal intensity value for each probe on each array. For each probe, the combined signal intensity values are combined across all arrays to provide an ordered sequence of probes from a lowest overall signal to a highest overall signal. The probes are then ranked according to the results of combining the combined signal intensity values, and binned into a plurality of bins. With regard to each probe, a metric representative of the multi-array distance of the signal intensities of the probe from a neutral expression value across all arrays is calculated and the probes are ranked within each bin based on the calculated metrics. Candidate dye-normalization probes may be selected by selecting at least one of the lowest ranked probes within each bin. Optionally, at least one of the lowest ranked probes in each bin may be discarded as outliers, and then at least one of the remaining lowest ranked probes may be selected from each bin as the candidate dye-normalization probes.

### BACKGROUND OF THE INVENTION

Molecular arrays are widely used and increasingly important tools for rapid hybridization analysis of sample solutions against hundreds or thousands of precisely ordered and positioned features containing different types of molecules within the molecular arrays. Molecular arrays are normally prepared by synthesizing or attaching a large number of molecular species to a chemically prepared substrate such as silicone, glass, or plastic. Each feature, or element, within the molecular array is defined to be a small, regularly-shaped region on the surface of the substrate. The features are typically arranged in a regular pattern. Each feature within the molecular array may contain a different molecular species, and the molecular species within a given feature may differ from the molecular species within the remaining features of the molecular array.

In one type of hybridization experiment, a sample solution containing radioactively, fluorescently, or chemoluminescently labeled molecules is applied to the surface of the molecular array. Certain of the labeled molecules in the sample solution may specifically bind to, or hybridize with, one or more of the different molecular species that together comprise the molecular array.

Following hybridization, the sample solution is removed by washing the surface of the molecular array with a buffer solution, and the molecular array is then analyzed by radiometric or optical methods to determine to which specific features of the molecular array the labeled molecules are bound. Thus, in a single experiment, a solution of labeled molecules can be screened for binding to hundreds or thousands of different molecular species that together comprise the molecular array. Molecular arrays commonly contain oligonucleotides or complementary deoxyribonucleic acid (“cDNA”) molecules to which labeled deoxyribonucleic acid (“DNA”) and ribonucleic acid (“RNA”) molecules bind via sequence-specific hybridization.

Generally, radiometric or optical analysis of the molecular array produces a scanned image consisting of a two-dimensional matrix, or grid, of pixels, each pixel having one or more intensity values corresponding to one or more signals.

Scanned images are commonly produced electronically by optical or radiometric scanners and the resulting two-dimensional matrix of pixels is stored in computer memory or on a non-volatile storage device. Alternatively, analog methods of analysis, such as photography, can be used to produce continuous images of a molecular array that can be then digitized by a scanning device and stored in computer memory or in a computer storage device.

In order to interpret the scanned image resulting from optical or radiometric analysis of a molecular array, the scanned image needs to be processed to locate the positions of features and extract data from the features. The extracted data may be further processed, for example to subtract background signal levels, and to normalize signals produced from different types of analysis. For example, dye normalization of optical scans conducted at different light wavelengths may need to be conducted to normalize different response curves produced by chromophores at different wavelengths. After normalization processing, ratios of the resultant signals may be determined for the features and further statistical processing of the signal ratios may be carried out to determine statistical significance of the results measured.

Currently practiced methodologies for dye normalization, such as the rank consistency method, for example, assume that for a given array, there are an equal number of up-regulated probes and down-regulated probes on the array at the time of optically analyzing, after hybridization and buffering, as described above, and that the mean of the distribution of the up-regulated and down-regulated expression ratios is zero. Many normalization procedures make such assumptions or similar assumption to these, and thus do not allow for a biased signal distribution in a sample set or results of an array. While such assumptions may be adequate for dye normalization of results from some large scale arrays, the risk of such assumptions becoming bad assumptions upon which to base a normalization technique increases as the size of the microarray (i.e., number of features on the array) becomes smaller.

For example, multiple small arrays may be provided on a single slide to allow multiple experiments to be processed simultaneously, under the same conditions, but with regard to fewer probes, e.g., to run more focused experiments with the advantages of less cost, as compared to having to use a large array for each experiment, and time savings, since multiple experiments can all be run on a single slide. However, with a smaller population of probes, that may be more focused on particular sequences, the assumption regarding an equal distribution of up-regulated and down-regulated probes on such an array, when normalizing the data, is statistically less valid and thus may give erroneous results. Moreover, probes selected for such focused experiments are often chosen by criteria involving their responses to a stimuli, environmental conditions, or other more inherent differences in the samples. Microarrays with small feature or probe counts will likely not span as broad a population of potential probes from which probes can be selected for normalization purposes, when compared to larger format microarrays, upon which current normalization techniques are designed to operate. Experimental probes on such a microarray with small feature counts may be inherently skewed to show a predominance in one dye channel versus another. In such an instance, use of a dye-normalization technique that assumes that there are an approximately equal number of up- and down-regulated probes will give erroneous results, e.g., dye biases.

There is a need for dye-normalization methodologies that are accurate for both large and small microarrays, and which do not rely upon assumptions that the distribution of the intensity log expression ratios to have a mean or median of zero. There is a need for normalization methodologies that yield accurate microarray results on a single microarray where the expression ratios of the biological probes are not evenly distributed about a mean or median of zero.

### SUMMARY OF THE INVENTION

Methods, systems and computer readable media for identifying dye-normalization probes. Intensity signals read from probes on a set of existing multi-channel microarrays are provided. The intensity signals are combined from each channel for each probe to generate a combined signal intensity value. For each probe, the combined signal intensity values are further combined across all arrays to provide and ordered sequence of probes from a lowest overall signal to a highest overall signal. The probes are then ranked according to the results of combining to form the ordered sequence of probes, and binned into a plurality of bins. With regard to each probe, a metric representative of the multi-array distance of the signal intensities of the probe from a neutral expression value across all arrays is calculated and the probes are ranked within each bin based on the calculated metrics. From such binning, candidate dye-normalization probes may be selected by selecting at least the lowest ranked probes within each bin. Optionally, at least the lowest ranked probe from each bin may be discarded as outliers, and then at least the lowest ranked of the remaining probes may be selected from each bin as the candidate dye-normalization probes.

The present invention also covers forwarding, transmitting and/or receiving results from any of the methods described herein.

These and other advantages and features of the invention will become apparent to those persons skilled in the art upon reading the details of the methods, systems and computer readable media as more fully described below.

### BRIEF DESCRIPTION OF THE DRAWINGS

FIG. 1 is an illustration of a plot of expression values read from a two-channel array.

FIG. 2A is a flow chart outlining exemplary steps that may be carried out in identifying candidate probes for dye normalization according to the present invention.

FIG. 2B illustrates the means of the absolute values of the LogRatios against the RankSumVector of the probes plotted across all arrays considered.

FIG. 3A outlines steps that may be taken for validating candidate normalization probes that have been selected.

FIG. 3B is a plot of average LogRatio vs. Average Log magnitude that may be preformed during a validation process.

FIG. 4 is a flow chart outlining exemplary steps that may be carried out in identifying candidate probes for dye normalization after employing previously selected candidates as normalization probes in an additional set of microarrays.

FIG. 5 shows plots of the distribution of LogRatios for all probes for a training data set processed, the distribution of the LogRatios read from the normalization probes selected from the training set, the distribution of LogRatios for all probes for a validation data set processed, and the distribution of the LogRatios read from normalization probes used in processing the validation set.

FIG. 6 is a block diagram illustrating an example of a generic computer system which may be used in implementing the present invention.

### DETAILED DESCRIPTION OF THE INVENTION

Before the present methods, systems and computer readable media are described, it is to be understood that this invention is not limited to particular hardware, software, microarrays or data sets described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a probe” includes a plurality of such probes and reference to “the array” includes reference to one or more arrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. . For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source.

An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups).

A nucleotide “probe” means a nucleotide which hybridizes in a specific manner to a nucleotide target sequence (e.g. a consensus region or an expressed transcript of a gene of interest).

An “array” or “microarray”, unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one that is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location.

A “large array” refers to an array containing at least about 10,000 features or probes.

A “small array” refers to an array containing a fewer number of features or probes than a large array, generally less than half the number of features/probes of a large array, and may have a number of features/probes that is an order of magnitude or more less than the number of features/probes on a large array.

“Hybridizing” and “binding”, with respect to polynucleotides, are used interchangeably.

The term “LogRatio” refers to the log (in any base, typically base 10 or base 2) of the ration of two signals, typically referring to two signals read from a microarray feature/probe. The two signals may be read from two channels of a microarray with regard to the same probe and may be signals of raw intensity, signals from which background level has been subtracted, or dye normalized signals, etc.

The term “fold change” refers to the difference or change in signals between two samples corresponding to a ration change from a neutrally expressed value.

The fold change is defined as positive if signal/channel one is greater than signal/channel two, and has a magnitude equal to the ratio of the signals of channel one/channel two. The fold change is defined as negative if signal/channel one is greater than signal/channel two, and has a magnitude equal to the ratio of the signals of channel two/channel one.

A “pulse jet” is a device which can dispense drops in the formation of an array. Pulse jets operate by delivering a pulse of pressure to liquid adjacent an outlet or orifice such that a drop will be dispensed therefrom (for example, by a piezoelectric or thermoelectric element positioned in a same chamber as the orifice). An array may be blocked into subarrays which may be hybridized as separate units or hybridized together as one array.

Any given substrate may carry one, two, four, eight or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features), each feature typically being of a homogeneous composition within the feature. Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used,. It will be appreciated though, that the interfeature areas, when present, could be of various sizes and configurations.

Each array may cover an area of, for example, less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample (for example, a fluorescently labeled polynucleotide or protein containing sample), and the array is then read. Reading of the array may be accomplished by illuminating the array and reading the location and intensity of resulting fluorescence at multiple regions on each feature of the array. For example, a scanner may be used for this purpose that is similar to the AGILENT MICROARRAY SCANNER manufactured by Agilent Technologies, Palo Alto, Calif. Other suitable apparatus and methods are described in U.S. patent applications Ser. No. 10/087447 “Reading Dry Chemical Arrays Through The Substrate” by Corson et al.; and in U.S. Pat. No. 6,518,556; 6,486,457; 6,406,849; 6,371,370; 6,355,921; 6,320,196; 6,251,685; and 6,222,664. The above patents and patent applications are incorporated herein by reference. Arrays may also be read by other methods or apparatus than the foregoing, with other reading methods, including other optical techniques (for example, detecting chemiluminescent or electroluminescent labels) or electrical techniques (where each feature is provided with an electrode to detect hybridization at that feature in a manner disclosed in U.S. Pat. No. 6,251,685, U.S. Pat. No. 6,221,583 and elsewhere). A result obtained from the reading may be used in accordance with the techniques of the present invention in screening and finding multiple drug treatment therapies. A result of the reading (whether further processed or not) may be forwarded (such as by communication) to a remote location if desired, and received there for further use (such as further processing).

When one item is indicated as being “remote” from another, this is referenced that the two items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

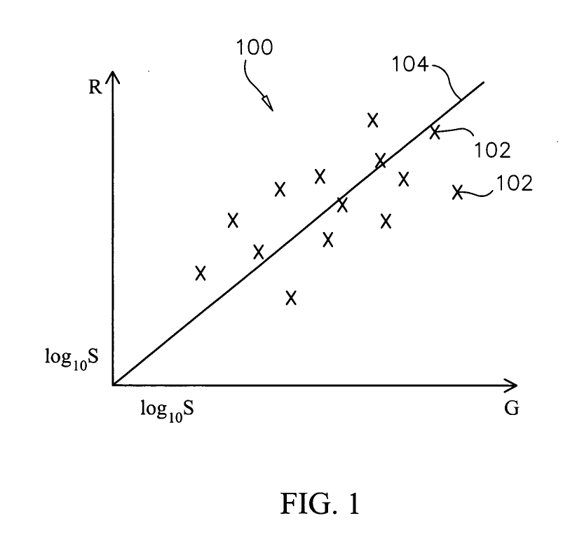

Referring now to FIG. 1, an illustration of a plot 100 of expression values read from a two-channel array (having red, “R” channel and green “G” channel) is shown. The x's 102 on the plot are representative expression values; in practice a much larger number of expression values typically appear on such a plot, depending upon the number of probes on the array being read and the number of those probes that are expressed. The expression values are typically plotted in a logarithmic scale (such as log₁₀ or log₂, for example), although this is not required. Thus, what is shown in FIG. 1 are the expression values for probes plotted as log₁₀ of the green channel signals (log₁₀G) versus log10 of the red channel (log₁₀R). The least differentially regulated probe signals (i.e., those signals that are the least up- or down- regulated) are those that are on or nearest to the diagonal 104 or a curve approximating diagonal 104, and the probes represented by these signals are typically the best candidates for dye-normalization reference probes.

Optimally, probes that exhibit signals that are neither up- or down-regulated across a set of experiments or arrays are those probes that are sought after for use as dye normalization reference probes. There are different approaches to development of normalization references. One approach is to prepare a reference to have as many probes expressed as possible. This approach is generally taken to form a “universal reference” and such a reference is generally constructed with probes from a variety of different types of tissue samples so that in any of a variety of different tissues, the normalization probes will be expressed (i.e., above the level of detectability by the system interpreting the probe signals). Such a reference is also sometimes referred to as a “far reference” in that it generally is not that similar to the sample tissue that is being examined in the experimental microarray. For example, the tissue being studied or experimented upon may be heart tissue, while a universal reference may contain heart tissue as only one of ten (or even zero of ten) tissues from which the reference was constructed.

Another approach, which generally tends to provide more reliable reference values, is to construct a “near reference”, in which the dye normalization probes are selected from tissues which are as close as possible to the experimental tissues that are to be measured against the reference probes. The nearest reference that can be constructed can be obtained by making a pool of all experimental samples that are to be analyzed, by taking a small amount of each sample and mixing them together, and then selecting reference probes from this mixture. However, in the diagnostic field, this approach is generally not possible, and the diagnostician must rely upon references generated from historical samples that have already been processed. However, banks of tissue samples are often stored, and may be similar enough to a sample to be analyzed so that pooling can be performed to provide a near reference. For example, a pool of fifty different existing/stored tumor tissue samples may be carried out to generate a near reference for a new tumor tissue sample to be analyzed.

In working with pooled tissues, the approach taken is to select the probes whose expression values are the closest to the line 104 indicating no differential regulation, across as many samples in the pool as possible. For each different array that these probes are included in, however, the expression values for these probes will vary somewhat. Accordingly, it is desirable to select probes which have the least amount of variation from neutral expression (from the diagonal or curve 104) to provide the most consistent reference across a plurality of arrays/samples.

As noted above, currently existing dye-normalization methods, such as the rank consistency method, for example (e.g., see AGILENT Feature Extraction Software (v. 7.5) User Manual, p. 223), while suitable for large arrays (e.g., array s containing about 11,000 features, 22,000 features, 44,000 features, or more) are not suitable for dye normalization of small arrays, which typically have a number of probes that is an order of magnitude smaller than the number of probes/features on a large array. For example, the Agilent 8-pack slide (Agilent Technologies, Inc., Palo Alto, California) has eight arrays on a single substrate, with each array having only about 1,900 features/probes. Such arrays with a relatively small number of features do not span the same population of potential probes from which a selection of dye-normalization probes can be made, when compared with those made available by the large arrays. Further, since the experimental probes on a small array may be inherently skewed to show predominance in one dye channel versus the other, the basis assumption relied upon by many existing dye normalization methodologies, i.e., that there are approximately equal numbers of up- and down- regulated probes, is not valid. The current techniques do not rely upon such assumption, and are thus applicable to small arrays a well as large arrays.

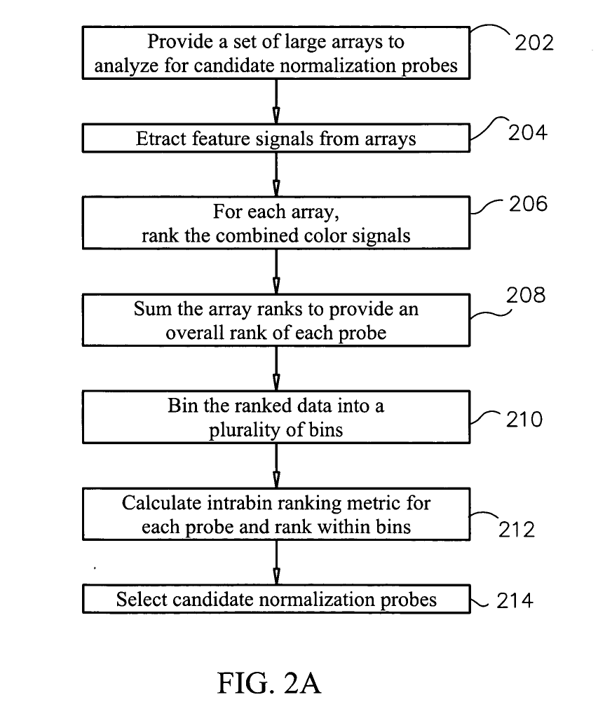

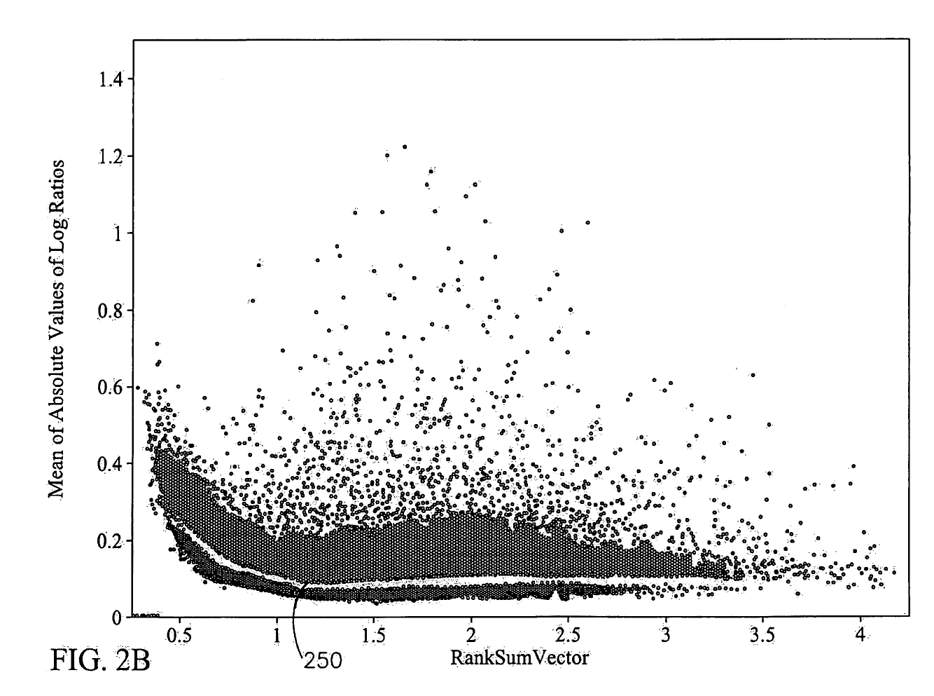

FIG. 2A is a flow chart outlining exemplary steps that may be carried out in identifying candidate probes for dye normalization. As noted, such procedure is well suited for providing dye normalization probes for use in small arrays, and therefore the example is described that way. However, these procedures are also applicable for providing dye normalization probes for large arrays. At step 202, a set of large arrays is provided for extraction of signals and processing to identify the candidate normalization probes. As mentioned above, such arrays may contain about eleven thousand features each, about twenty-two thousand features each, about forty-four thousand features each, or more, or some other number of features that is at least about an order of magnitude larger than the number that the experimental arrays contain, for purposes of identifying normalization probes for small arrays.

The arrays provided in step 202 preferably contain data representing the same kinds of tissue or cell line samples to be investigated in the experimental arrays for which dye neutralization probes are sought. Additionally, the same labeling, hybridization and wash protocols are desired to improve the chances of identifying well performing dye neutralization probes. However, if these conditions cannot be met, efforts should be taken to choose arrays that are as close as possible to the actual experimental biological arrays to be studied.

At least one or two pairs of dye swap experiments may optionally be included in the arrays provided in step 202, as such data may be useful for validating results. The use of dye swap pairs helps to remove biological and technological biases from the probe selection process. The set should include significant numbers of differentially expressed probes/genes, and as such, should not include a significant number of self-self hybridizations, since these will tend to yield non-differentially expressed data.

Feature signals are then extracted from the arrays (step 204) for further processing. Features may be identified using techniques such as provided in any or all of U.S. Pat. No. 6,591,196 copending, commonly owned application Ser. no. 10/449,175 filed May 30, 2003, titled “Feature Extraction Methods and Systems” and copending, commonly owned application no. (application Ser. No. not yet assigned, Attorney's Docket No. 10040225-1) filed Jun. 16, 2004, titled “System and Method of Automated Processing of Multiple Microarray Images” and/or by use of a feature extraction system using Agilent Feature Extraction Software (Agilent Technologies, Inc., Palo Alto, Calif.), for example. Alternatively, feature signals may simply be provided to the system as the initial datasets to work with.

For each array that features are extracted from, the combined color signals from each feature for that array are then ranked (e.g., assigned a rank from lowest to highest signal strength). For example, a combined color signal may be determined by calculating the geometric mean of the red and green signals for a probe, i.e., combined color signal=(red signal *green signal)^{1/2} or a logarithmic of these values may be used. Further, other alternative metrics may be employed, such as a Euclidean distance metric, a straight mean metric, or logarithmic or either of these metrics, for example. Surrogate features and saturated features are typically not considered from this stage forward. Surrogate probes are typically used for negative background subtracted signals and also for signals that are not significantly above the background level in either channel, and as such can be discounted, ab initio, from consideration as possible normalization probe candidates. Saturated features can be similarly discounted, as not providing a reliable signal level reading. Control probes are generally also not considered for possible normalization candidates as they are not biological in nature.

It is desirable to span the space of the expression values are that observed, so that normalization probes defining a normalization curve are represented over the entire range of the expression values identified in the array sets. By doing so, this provides a more accurate normalization curve over the entire range of potential expression values that are likely to be encountered when measuring experimental data arrays for similar tissue types under similar processing conditions.

For each probe considered, the ranks corresponding to each of the arrays for that probe are summed at step 208 to provide an overall rank for each probe across all arrays, which is also referred to a “RankSumVector”. Alternatively, signals across arrays may be combined by a measure other than RankSumVector. For example, the rank of sum (or median or mean) of all signals for each probe may be calculated. Other metrics may also be used, which result in ranking or ordering the probes from the weakest to the strongest signals. The probes are next binned into a predetermined number of bins so that each bin typically includes approximately the same number or probes. The number of bins chosen may be selected by the user depending upon how many representative locations along the normalization line it is desired to have probes located. It is important to span the signal space of the biological probes. If too few bins are considered, then the top or bottom of the dynamic range may be underrepresented (i.e., by too few probes). However, bins of varying size may be used alternatively if desired, if the distribution of probes is such that it is not substantially evenly spaced over the signal space. Further alternatively, bins may be identified across the signal axis to divide the probes in the set (typically these bins are of equal size, i.e., each containing approximately the same number of points/probes; alternatively, they may cover an equal range of signal intensity on a log scale, although equal size bins are also not necessary using this technique either). An advantage of using equally sized bins is that each bin has roughly the same population statistics.

The mean (or other representative metric such as the sum) of the absolute values of the LogRatios (or fold-change) is next computed (e.g., mean(abs(LogRatio))) at step 212. Further alternatively, a metric involving both the LogRatios and some measure of noise, such as standard deviation, variance, interquartile range, or the like may be used as the ranking metric. The calculated metrics (sums or means of absolute values of the LogRatios, with or without noise factor) are then ranked within each bin. Alternatively, LogRatio and noise metrics may be initially considered separately, with one set of ranks being assigned to the LogRatio metrics and another set of ranks being assigned to the noise metrics. Then the two sets of ranks can be combined to provide an overall rank or score that reflects both the LogRatio metrics and the noise metrics.

At step 414, candidate normalization probes are selected from each ranked bin by selecting a predetermined number of the lowest ranked probes from each bin (i.e., with the lowest average or sum of absolute values of LogRatios). The number of probes selected from each bin will depend upon the available real estate on the arrays for which they will be used. The available real estate depends on such factors as the total number of features available on an array, the number of signature probes to be placed on the array, the number of quality control probes to be included, etc. Thus, for example, for use on arrays having 1,900 features, it may be desirable to pick two to five probes from each bin when the number of bins used is two hundred, resulting in four hundred to one thousand normalization probes. The actual number of normalization probes used will vary depending upon the confidence level desired for measurements taken from the arrays on which they are used.

FIG. 2B illustrates the means of the absolute values of the LogRatios against the RankSumVector of the probes plotted across all arrays considered. Those points below line 250 represent the probes that were selected as dye normalization probes, and those points above line 250 represent all other probes from the arrays considered.

Rather than selecting the absolute lowest ranked probes in step 214, the method may be modified so as to discard a predetermined number of the lowest ranked probes. In some instances it has been observed that the very lowest data points in each bin may be outliers. Optionally then, a robust version of the above-described process discards a predetermined number of the lowest data points in each bin at step 214 and selects the next lowest data points as representing the selected probes for dye normalization. For example, when running the process on two hundred bins with 100 to 250 probes per bin, the robust process may discard the lowest four data points and select the next five lowest data points in each bin. Of course, the predetermined numbers for discarding and selecting are variable and will be determined, at least in part, by the real estate of the arrays to which the normalization probes are to be applied.

Once the candidate normalization probes have been selected by any of the techniques described above, the selected probe set may be applied in the experimental data arrays for dye-normalization thereof. Optionally, however, the candidate normalization probes may first be subjected to a validation process. When a validation procedure is to be performed, the initial data from the set of large arrays provided originally is divided by randomly dividing the set of arrays into two subsets of arrays, a training subset and a validation subset. While such division may be done on a “whole array basis”, i.e., for each array, assigning the entire array either to the training set or the validation set, an even better separation for validation purposes is to separate according to samples, by excluding all replicates or dye swaps of some set of samples, while including the replicates and dye swaps for all other samples not in the defined set.

As noted, the validation processing is optional and therefore not necessary. Validation processing is used primarily to validate the selection of normalization probes. Once validated, processing is likely somewhat more robust if all arrays or experiments are used in the selection of normalization probes.

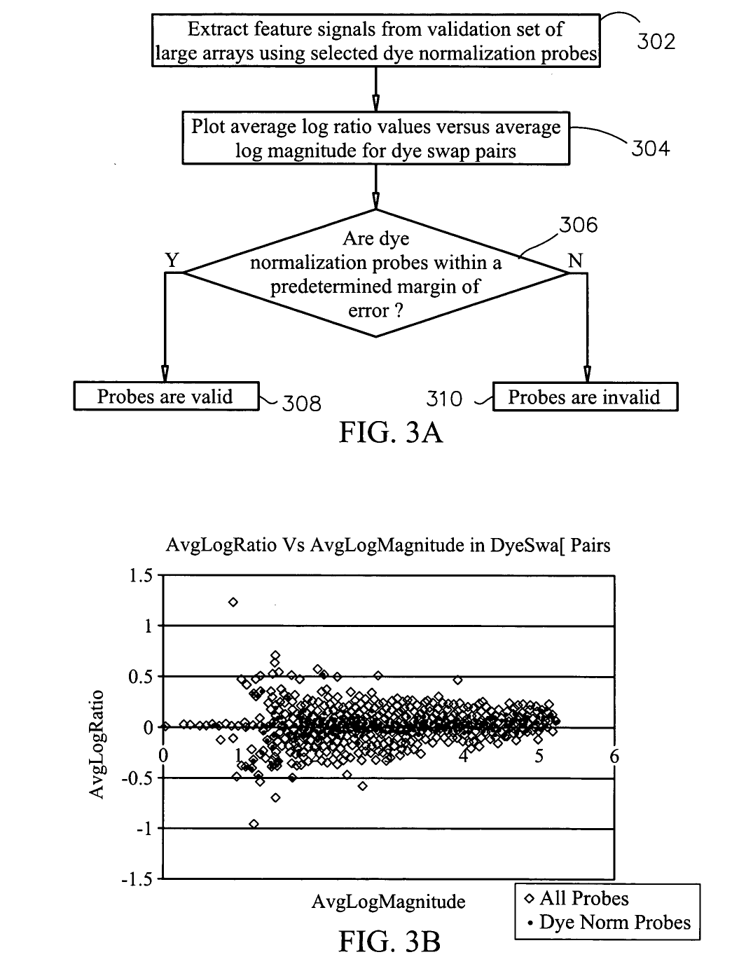

During validation processing, the training subset is used for carrying out the process described above with regard to FIG. 2A to identify the candidate normalization probes. Once so identified, the features of the validation subset are extracted in step 302 (FIG. 3A) using the selected candidate normalization probes to dye-normalize the results. With regard to dye-swap pairs of data (i.e., wherein one or more sample pairs (where a pair consists of a biological sample and a referenced sample (for the common reference approach), and/or biological pairs in round-robin or circular experiments), these pairs of data are run under the same conditions twice, but where the reference or first sample data channel is a first color (e.g., red) and the sample or second sample data channel is a second color (e.g., green) on the first run and vice versa on the second run), a plot 350 of average LogRatio vs. Average Log magnitude is made (step 304) as shown in FIG. 3B. Since the sum of LogRatios for each normalization probe from a dye swap pair should be zero (ideally, i.e., log of one being zero), the plot 350 should show the normalization probes 360 being substantially aligned with the zero average log ratio level for valid dye-normalization probes, while probes 370 that are differentially expressed are scattered about, but close to the line at zero average log ratio line. A threshold may be predefined for rejecting those probes that are “bad” (e.g., when probes are included that have too large a dye-bias). For example, if the absolute value of the LogRatio is above some threshold (e.g., two times the standard deviations of all probes on the array or within the same bin) on more than some number or percentage of experiments or arrays, then the probe may be rejected as being significantly regulated (or altered) in some samples. The number or percentage of experiments against which failure may be judged (margin of error) will vary, as this depends upon the diversity of the samples within the set. For example, a set of samples may involve rarely expressed, but important genes, or rare genes that are expressed at very high copy number when they are switched on. However, it may be the case that the rarely high signal corresponds to some technical artifact on a small number of arrays (e.g., a dust speck). As a general rule, the percentage of experiments or arrays as the threshold for the margin of error may be stated as somewhat less than 5% to about 10%, while keeping in mind that the characterization of the samples may significantly alter such range, for reasons noted, or for other characterizations that make the samples out of the ordinary.

If the validation process determines that the normalization probes are not within a predetermined margin of error at step 306, such as in a manner as described in the preceding paragraph, for example, then the probes are considered to be invalid at step 310 and another round of the probe selection process will need to be carried out. There may be various reasons why a failure would be determined (i.e., declaration of invalidity) at step 310. One is that, contrary to the underlying assumption, the arrays or samples that were used in the training set were not diverse enough to span the diversity of the samples in the validation set. In this case, another training set will need to be selected that is more diverse, possibly by using a broader set of samples and/or larger number of arrays for probe selection. Another reason may be that the diversity of probe expression across the set of samples is such that there are no probes that effectively behave as normalization probes. This means that all probes are equivalently changed in expression levels across the sample set and that therefore no subset of probes will be more valid than any other subset of probes for use as normalization probes. This second case is very unlikely, as there are generally some probes that are more diverse in their expression patterns than others, unless they are specifically selected in a manner that enforces uniform diversity.

If, on the other hand, the process determines that the normalization probes are within the margin of error, then the probes are determined to be valid at step 308 and may be used in the experimental data arrays for dye normalization purposes.

Upon adding the validated dye normalization probes to one or more arrays to be evaluated (step 402) (such as a small array containing experimental data or other sample, for example) feature signals are extracted from the arrays to which the normalization probes have been added at step 404, and dye normalization is carried out based on the added dye normalization probes (i.e., the selected normalization probe set).

Optionally, after feature extracting the arrays, the signal data may be processed similarly to the procedure described above with regard to FIG. 2A.

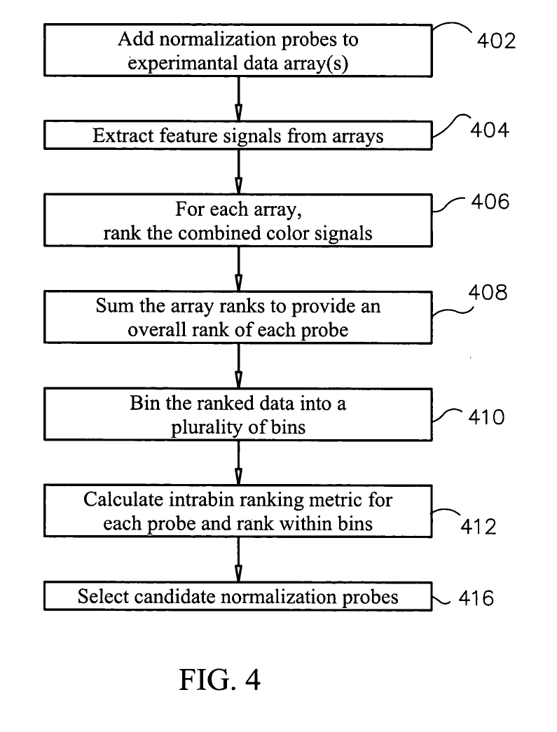

That is, for each array the combined color signals (e.g., geometric means) may be ranked at step 406. Then the array ranks may be summed at step 408 to provide an overall rank of each probe at step 408. At step 410, the ranked data may be grouped into a predetermined number of bins. In examples where the arrays used this time are small arrays, the number of bins used will generally be smaller than when processing a large number of probes from large arrays, as described with regard to FIG. 2A. For example, if the arrays used in the process described with regard to FIG. 2A each include twenty-two thousand features and the number of bins employed was two hundred, while the arrays considered in the process with regard to FIG. 4 each include only about one thousand nine hundred features, the number of bins used may be about sixty. As noted before, however, the number of bins chosen may vary.

At step 412, an intrabin ranking metric (such as sum or mean of the absolute values of the LogRatios, for example) is calculated for each probe across all arrays considered, and then the probes are ranked within bins according to the ranking metrics, and a predetermined number of the lowest ranking (or lowest ranking after discarding a predetermined number of the previously lowest ranking probes when carrying out the robust option) probes are selected as dye-normalization probes in the same manner as that described above with the process described in FIG. 2A. Again, these predetermined numbers may be selected by the operator of the process and may vary depending on factors which may include, but are not limited to, the target number of normalization probes expected to be used, the real estate available on the arrays on which the normalization probes are to be used, etc. For the example provided above (i.e., where the arrays each contain one thousand nine hundred features) and where the number of bins used is sixty, the robust process may discard the lowest two probes from each bin and select the third through the seventh ranked probes in each bin to provide three hundred dye normalization probes, for example.

These finally selected probes should be a subset of the probes that were selected in the process of FIG. 2A at step 214. Optionally, the data from the arrays may be randomly divided prior to extraction at step 404 into training and validation subsets in the same manner described above with regard to FIGS. 2A and 3. In this case, the training set would be processed at steps 404 to 414, and then the validation set would be used to validate the selected probes from step 414 by processing in the same way as described above with regard to FIG. 3A. That is, feature signals of the validation set would be extracted and dye-normalized using the probes selected at step 414, and then the average LogRatio values of the signals generated by the extracted features would be plotted against average log magnitudes of the same for dye swap pairs. If the plot of the dye normalization probes is within a predetermined margin of error, then the probes are determined to be valid.

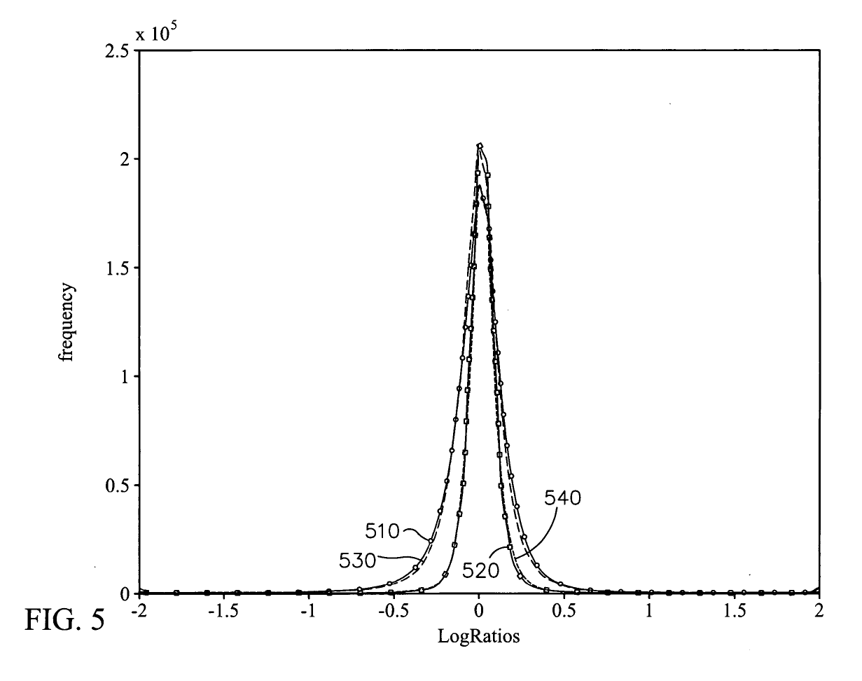

FIG. 5 shows a plot 510 of the distribution of LogRatios for all probes for the training data set processed with regard to FIG. 4 and a plot 520 of the distribution of the LogRatios read from the normalization probes selected from the training set are shown. Additionally, a plot 530 of the distribution of LogRatios for all probes for the validation data set processed with regard to the arrays described with regard to the validation set corresponding to the arrays processed in FIG. 4 are shown and a plot 540 of the distribution of the LogRatios read from normalization probes used in processing the validation set is shown.

The close correlation of these plots further confirms the validity of the use of the selected probes for dye normalization purposes.

Using the normalization probes identified by any of the above described techniques, dye-normalized expression ratios may then be computed by feature extraction software. For example, many feature extraction systems use algorithms for filter and smoothing, such as LOESS, or LOWESS, for example to standardize the normalization line based on the normalization probes and to computer expression ratio readings for other probes that are differentially expressed. For example, such systems, for each array, take the normalization probes which are closest to the diagonal and normalize them to fit to the diagonal/curve that indicates no differential expression; then calculate log ratios of differentially expressed probes relative to the normalization curve.

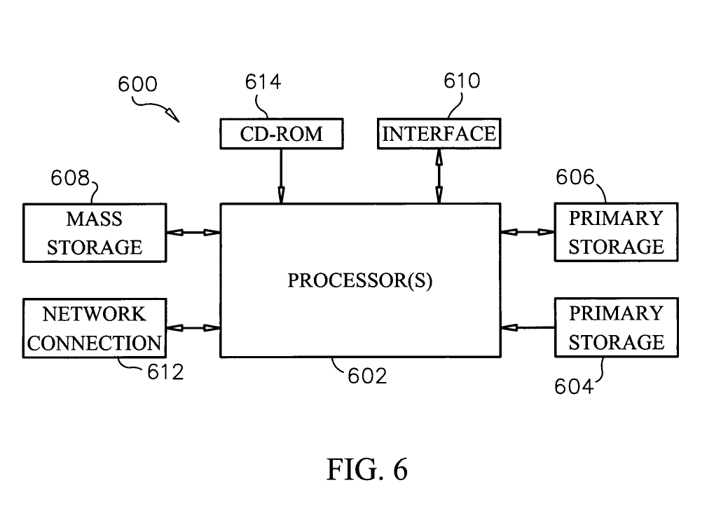

FIG. 6 illustrates a typical computer system 600 that may be used in processing events described herein. The computer system 600 includes any number of processors 602 (also referred to as central processing units, or CPUs) that are coupled to storage devices including primary storage 606 (typically a random access memory, or RAM), primary storage 604 (typically a read only memory, or ROM). As is well known in the art, primary storage 604 acts to transfer data and instructions uni-directionally to the CPU and primary storage 606 is used typically to transfer data and instructions in a bi-directional manner Both of these primary storage devices may include any suitable computer-readable media such as those described above. A mass storage device 608 is also coupled bi-directionally to CPU 602 and provides additional data storage capacity and may include any of the computer-readable media described above.

Mass storage device 608 may be used to store programs, data and the like and is typically a secondary storage medium such as a hard disk that is slower than primary storage. It will be appreciated that the information retained within the mass storage device 608, may, in appropriate cases, be incorporated in standard fashion as part of primary storage 606 as virtual memory. A specific mass storage device such as a CD-ROM 614 (or DVD-ROM, CD-RW, DVD-RW, or the like) may also pass data uni-directionally to the CPU.

CPU 602 is also coupled to an interface 610 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 602 optionally may be coupled to a computer or telecommunications network using a network connection as shown generally at 612. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for calculating means of absolute values of LogRatios for each probe may be stored on mass storage device 608 or 614 and executed on CPU 608 in conjunction with primary memory 606.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CD-RW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of identifying normalization probes from a set of existing microarrays for use as dye-normalization probes in other microarrays, said method comprising the steps of:
providing intensity signals read from probes on a set of multi-channel microarrays;
combining the signals from each channel for each probe to generate a combined signal intensity value for each probe on each array;
for each probe, combining the combined signal intensity values across the arrays to provide an ordered sequence of probes from a lowest overall signal to a highest overall signal;
ranking the probes according to results from said combining step;
binning the ranked probes into a plurality of bins;
with regard to each probe, calculating a metric representative of a multi-array distance of the signal intensities of the probe from a neutral expression value across all arrays;
ranking the probes within each bin based on the calculated metrics representative of the average distance of the signal intensities of the probes from the neutral expression value; and
selecting at least one of the lowest ranked probes within each bin.

2. The method of claim 1, further comprising ranking the combined signal intensity values with regard to each array; wherein said combining the combined signal intensity values across the arrays comprises combining the ranks of the combined signal intensity values.

3. The method of claim 2, wherein said combining the ranks of the combined signal intensity values comprises: for each probe, summing the ranks of that probe across all arrays to provide a RankSumVector for each probe.

4. The method of claim 1, further comprising discarding at least one of the lowest ranked probes within each bin, after said ranking the probes within each bin based on the calculated metrics representative of the multi-array distance of the signal intensities of the probes from the neutral expression value, wherein said selecting at least one of the lowest ranked probes within each bin selects the at least one of the lowest ranked probes remaining after discarding said at least one of the lowest ranked probes.

5. The method of claim 1, further comprising applying said selected probes as normalization probes within at least one additional microarray for use as dye normalization probes.

6. The method of claim 5, further comprising processing said at least one additional microarray to obtain signal intensity readings from probes on said at least one additional microarray; and normalizing said signal intensity readings with respect to dye bias, based on said normalization probes.

7. The method of claim 5, wherein said set of existing microarrays are large arrays and wherein said at least one additional microarray is a small array.

8. The method of claim 1, wherein said binning comprises placing a substantially equal number of probes into each of said predetermined number of bins.

9. The method of claim 1, wherein said combining comprises calculating a geometric mean of the channel signals.

10. The method of claim 1, wherein said calculating a metric comprises calculating at least one of: the sum of the absolute values of the LogRatios or fold changes of the probe across all arrays and the mean of the absolute values of the LogRatios or fold changes of the probe across all arrays.

11. The method of claim 1, wherein said calculating a metric includes calculation of noise factors.

12. The method of claim 10, wherein said calculating a metric further includes calculation of noise factors.

13. The method of claim 1, wherein the intensity signals provided are from a training set of the existing microarrays, wherein said method further comprises
randomly dividing said set of existing microarrays into a training set of microarray and a validation set of microarrays, prior to said providing intensity signals, wherein said validation and training sets each include a plurality of dye-swap pairs of data, and wherein said intensity signals are read from probes on said training set of microarrays.

14. The method of claim 13, further comprising the steps of:
providing intensity signals read from probes on said validation set of microarrays and dye-normalizing said intensity signals from said probes on said validation set based on use of said probes selected in claim 1 as normalization probes;
calculating average LogRatio or fold change values and average Log magnitude values of said intensity signals for said dye-swap pairs;
plotting said average LogRatio or fold change values against said average Log magnitude values of said intensity signals for said dye-swap pairs; and
determining that said selected probes are valid dye-normalization probes if the plot of said average LogRatio or fold change values against said average Log magnitude values of said intensity signals for said dye-swap pairs is within a predetermined margin of error from an average LogRatio value of zero.

15. The method of claim 1, further comprising applying said selected probes as normalization probes within additional multi-channel microarrays for use as dye normalization probes;
feature extracting the probes of said additional microarrays to provide intensity signals read from probes on the additional microarrays;
combining the signals from each channel for each probe to generate a combined signal intensity value for each probe on each additional microarray;
for each probe, combining the combined signal intensity values across all additional microarrays to provide an ordered sequence of probes from a lowest overall signal to a highest overall signal;
ranking the probes according to results from said combining step;
binning the ranked probes into a plurality of bins;
with regard to each probe, calculating a metric representative of a multi-array distance of the signal intensities of the probe from a neutral expression value across all the additional arrays;
ranking the probes within each bin based on the calculated metrics representative of the average distance of the signal intensities of the probes from the neutral expression value; and
selecting at least one of the lowest ranked probes within each bin.

16. The method of claim 15, further comprising ranking the combined signal intensity values with regard to each additional array; wherein said combining the combined signal intensity values across the additional arrays comprises combining the ranks of the combined signal intensity values.

17. The method of claim 16, wherein said combining the ranks of the combined signal intensity values comprises: for each probe, summing the ranks of that probe across all additional arrays to provide a RankSumVector for each probe.

18. The method of claim 15, further comprising discarding a second predetermined number of the lowest ranked probes within each bin, after said ranking the probes within each bin based on the calculated metrics representative of the average distance of the signal intensities of the probes from the neutral expression value, wherein said selecting a predetermined number of the lowest ranked probes within each bin selects the predetermined number of the lowest ranked probes remaining after discarding said second predetermined number of the lowest ranked probes.

19. The method of claim 15, wherein said set of existing microarrays are large arrays and wherein said additional microarrays are small arrays.

20. The method of claim 15, wherein said lowest ranked probes selected in claim 13 are a subset of the lowest ranked probes selected from the set of existing microarrays.

21. The method of claim 15, wherein said feature extracting is performed with regard to a training set of the additional microarrays, wherein said method further comprises:
randomly dividing said additional microarrays into a training set of additional microarrays and a validation set of additional microarrays, prior to said feature extracting, wherein said validation and training sets of additional microarrays each include a plurality of dye-swap pairs of data, and wherein said feature extracting is performed on said training set of additional microarrays.

22. The method of claim 21, further comprising the steps of:
feature extracting probes on said validation set of addition microarrays to provide intensity signals therefore and dye-normalizing said intensity signals from said probes on said validation set based on use of said probes selected in claim 16 as normalization probes;
calculating average LogRatio or fold change values and average Log magnitude values of said intensity signals for said dye-swap pairs;
plotting said average LogRatio or fold change values against said average Log magnitude values of said intensity signals for said dye-swap pairs; and
determining that said selected probes are valid dye-normalization probes if the plot of said average LogRatio or fold change values against said average Log magnitude values of said intensity signals for said dye-swap pairs is within a predetermined margin of error from an average LogRatio value of zero.

23. A method comprising forwarding a result obtained from the method of claim 1 to a remote location.

24. A method comprising transmitting data representing a result obtained from the method of claim 1 to a remote location.

25. A method comprising receiving a result obtained from a method of claim 1 from a remote location.

26. A system for identifying normalization probes from a set of existing microarrays for use as dye-normalization probes in other microarrays, said system comprising:
means for interpreting intensity signals read from probes on a set of multi-channel microarrays;
means for combining the signals from each channel for each probe to generate a combined signal intensity value for each probe on each array;
for each probe, means for combining the combined signal intensity values across the arrays to provide an ordered sequence of probes from a lowest overall signal to a highest overall signal;
means for ranking the probes according to results from said combining step;
means for binning the ranked probes into a plurality of bins;
with regard to each probe, means for calculating a metric representative of a multi-array distance of the signal intensities of the probe from a neutral expression value across all arrays; and
means for ranking the probes within each bin based on the calculated metrics representative of the average distance of the signal intensities of the probes from the neutral expression value.

27. The system of claim 26, further comprising means for selecting at least one of the lowest ranked probes within each bin.

28. The system of claim 26, further comprising means for discarding at least one of the lowest ranked probes within each bin as outliers, after ranking the probes within each bin based on the calculated metrics representative of the multi-array distance of the signal intensities of the probes from the neutral expression value.

29. The system of claim 27, further comprising means for applying said selected probes as normalization probes within at least one additional microarray for use as dye normalization probes.

30. The system of claim 29, further comprising means for processing said at least one additional microarray to obtain signal intensity readings from probes on said at least one additional microarray; and means for normalizing said signal intensity readings with respect to dye bias, based on said normalization probes.

31. A system for identifying normalization probes from a set of existing large microarrays for use as dye-normalization probes in small microarrays, said system comprising:
means for interpreting intensity signals read from probes on a set of large multi-channel microarrays;
means for combining the signals from each channel for each probe to generate a combined signal intensity value for each probe on each large array;
for each probe, means for combining the combined signal intensity values across the large arrays to provide an ordered sequence of probes from a lowest overall signal to a highest overall signal;
means for ranking the probes according to results from said combining step;
means for binning the ranked probes into a plurality of bins;
with regard to each probe, means for calculating a metric representative of a multi-array distance of the signal intensities of the probe from a neutral expression value across all large arrays;
means for ranking the probes within each bin based on the calculated metrics representative of the average distance of the signal intensities of the probes from the neutral expression value;
means for selecting at least one of the lowest ranked probes within each bin; and
means for applying the selected probes as normalization probes within at least one small microarray for use as dye normalization probes.

32. A computer readable medium carrying one or more sequences of instructions for identifying normalization probes from intensity signals read from probes on a set of existing microarrays for use as dye-normalization probes in other microarrays, wherein execution of one or more sequences of instructions by one or more processors causes the one or more processors to perform the steps of:
combining the intensity signals from each channel for each probe to generate a combined signal intensity value for each probe on each array;
for each probe, combining the combined signal intensity values across the arrays to provide an ordered sequence of probes from a lowest overall signal to a highest overall signal;
ranking the probes according to results from said combining step;
binning the ranked probes into a plurality of bins;
with regard to each probe, calculating a metric representative of a multi-array distance of the signal intensities of the probe from a neutral expression value across all arrays; and
ranking the probes within each bin based on the calculated metrics representative of the average distance of the signal intensities of the probes from the neutral expression value

33. The computer readable medium of claim 32, wherein execution of one or more further sequences of instructions by one or more processors causes the one or more processors to perform the additional step of selecting at least one of the lowest ranked probes within each bin.
